(19) United States
(12) Patent Application Publication
(10) Pub. No.: US 2026/0345494 A1
(43) Pub. Date: Nov. 5, 2026

(54) PEDIATRIC FORMULATION OF TYROSINE KINASE INHIBITORS

(71) Applicant: YALE UNIVERSITY, New Haven, CT (US)

(72) Inventors: Lois B. Rosenberger, New Haven, CT (US); Gregory J. Kelso, New Haven, CT (US); Steve Rosenberger, New Haven, CT (US)

(21) Appl. No.: 19/636,558

(22) Filed: Apr. 1, 2026

Related U.S. Application Data
(63) Continuation of application No. 18/516,195, filed on Nov. 21, 2023, now abandoned.
Application No. 18/516,195 is a continuation of application No. 17/217,060, filed on Mar. 30, 2021, now abandoned.
(60) Provisional application No. 63/001,824, filed on Mar. 30, 2020.

(51) Int. Cl. A61K 9/16; A61K 9/50; A61K 31/506

(52) U.S. Cl. CPC A61K 9/1676; A61K 9/1611; A61K 9/1623; A61K 9/1652; A61K 9/1682; A61K 9/5042; A61K 9/5089; A61K 31/506

(57) ABSTRACT

A pediatric dosage form for dosing ultra-low doses of an active pharmaceutical ingredient having a plurality of pellets where each pellet consists essentially of a coating of tyrosine kinase inhibitor over a nonpareil seed. The tyrosine kinase inhibitor coated nonpareils are coated thereon with a coating consisting essentially of hydroxypropyl methylcellulose. The active coated beads according to the invention allows for human subject dosing on a mg/kg basis. The sum of such pellets contained within a capsule forming a single dosage unit of an ultra-low dose of tyrosine kinase inhibitor.

### CROSS REFERENCE TO RELATED APPLICATIONS

This application is a continuation of and claims priority and benefit of U.S. Non-Provisional patent application Ser. No. 18/516,195, filed on Nov. 21, 2023, which is a continuation of and claims priority and benefit of U.S. Non-Provisional patent application Ser. No. 17/217,060, filed on Mar. 30, 2021 and entitled “PEDIATRIC FORMULATION OF TYROSINE KINASE INHIBITORS”, which claims priority to and the benefit of U.S. Provisional Patent Application No. 63/001,824, filed on Mar. 30, 2020 and entitled “PEDIATRIC FORMULATION OF TYROSINE KINASE INHIBITORS”, the contents of which are incorporated herein by reference as though fully set forth herein.

### BACKGROUND OF INVENTION

Congenital heart disease (CHD) is the most common defect found in newborns, occurring in about 1% of live births. Over 1 million people in the United States have some form of CHD, most of whom require continual monitoring and treatment to prevent deterioration of cardiac function. Atrioventricular canal defect (AVCD) includes different anomalies of atrioventricular valves and atrial and ventricular septa. In the complete form, a single common atrioventricular valve and an atrial septal defect (ostium primum) confluent with a posterior ventricular septal defect in the inlet portion of the ventricular septum are found. In the partial form, there are two separate right and left atrioventricular valves with a clefted mitral valve, an atrial septal defect (ostium primum), and no ventricular septal communication. Cleft mitral valve is considered the less severe form of AVCD. AVCD is also the most common CHD found in children with Down syndrome and one of the structural heart defects most frequently associated with extra-cardiac anomalies in the setting of chromosomal and mendelian disorders. Distinct anatomic features are found in AVCD associated with Noonan Syndrome (NS). In general this defect is of the partial type, eventually associated with subaortic stenosis, due to accessory fibrous tissue and/or anomalous insertion of the mitral valve with anomalous papillary muscle of the left ventricle.

Congenital heart disease (CHD) occurs in approximately 60-86% of patients affected by a RASopathy, a group of disorders with abnormalities in the RAS-MAPK pathway. Pulmonary valve stenosis (PVS) and hypertrophic cardiomyopathy are the most common defects displaying a distinct association with the RASopathies. Many people with NS are born with some form of heart defect (congenital heart disease), accounting for some of the key signs and symptoms of the disorder. Some heart problems can occur later in life. Some forms of congenital heart disease associated with this disorder include valve disorders. Pulmonary valve stenosis is a narrowing of the pulmonary valve, the flap of tissue that separates the lower right chamber (ventricle) of the heart from the artery that supplies blood to the lungs (pulmonary artery). It is the most common heart problem seen with NS, and it may occur alone or with other heart defects. Additional cardiac disorders include thickening of the heart muscle (hypertrophic cardiomyopathy). This is abnormal growth or thickening of the heart muscle that affects some people with NS. Structural defects of the heart may be present in people with NS. The defects can involve a hole in the wall that separates the two lower chambers of the heart (ventricular septal defect), narrowing of the artery that carries blood to the lungs for oxygen (pulmonary artery stenosis), or narrowing of the major blood vessel (aorta) that carries blood from the heart to the body (aortic coarctation). Also, irregular heart rhythm occurs in the majority of people with NS. The spectrum of CHDs in NS with multiple lentigines (NSML) is wider, and the family of atrioventricular canal defects (AVCD) is the third most common heart defect. Most patients with cardiovascular disease and RASopathy-associated congenital heart disease need treatment for many years. In particular, RASopathy-associated congenital heart disease are usually associated with low mortality rates.

Unfortunately, there is no cure for NS. Current treatment is aimed at managing the signs and symptoms of the disorder. NS is a lifelong disorder; the severity of the heart defects determine the life expectancy of an individual. Therefore, a need exists for a treatment of this cardiovascular disease in patients with low-risk therapies having maximal effect on heart disease found in NS.

### SUMMARY OF INVENTION

As described below, the present invention includes compositions and pharmaceutical formulations to inhibit aberrant protein tyrosine phosphorylation, such as phosphorylation of Src family tyrosine kinases and their substrates.

In one aspect, the invention includes a pediatric formulation of treating a cardiovascular disease or condition having aberrant protein tyrosine phosphorylation in a subject, comprising administering an ultra-low dosage of a tyrosine kinase inhibitor (TKI) to a subject in need thereof, wherein the TKI decreases aberrant levels of tyrosine phosphorylation and improves at least one cardiac function in the subject.

In another aspect, the invention includes a pharmaceutical formulation comprising an ultra-low dose of a TKI as described herein and a pharmaceutically acceptable carrier. In various embodiments of the above aspects or any other aspect of the invention delineated herein, the congenital heart disease is associated with a RASopathy, such as a RASopathy selected from the group consisting of Neurofibromatosis Type 1, Noonan syndrome, Noonan syndrome with multiple lentigines (Leopard syndrome), capillary malformation-arteriovenous malformation syndrome, Costello syndrome, cardio-facio-cutaneous syndrome, and Legius syndrome.

In one illustrative embodiment, the ultra-low dosage of the TKI is in the range of about 175 fold to about 250 fold lower than a chemotherapeutic dosage of the TKI.

In another embodiment, the TKI is selected from the group consisting of afatinib, axitinib, bosutinib, cabozantinib, cediranib, ceritinib, crizotinib, dabrafenib, dasatinib, erlotinib, everolimus, gefitinib, ibrutinib, imatinib, lapatinib, lenvatinib, lestaurtinib, nilotinib, nintedanib, palbociclib, pazopanib, ponatinib,

In a further embodiment a low dose coated dastinib formulation is disclosed is for use in pediatric patients. The manufacturing process of the pediatric formulation allows for subject dosing on mg/kg basis. The manufacturing process is a Wurster coating process on nonpareils; nonpareils are small beads made up of various substrates. The nonpareils, typically sugar seeds having a mesh size of about 20 to about 50.

In another illustrative embodiment the nonpareils are coated with more than an equal amount by weight of a TKI compound, which in one illustrative embodiment is anhydrous dastinib.

In a further illustrative embodiment, nonpareil seeds are placed in a coating pan and wetted with a 30% sucrose solution with the aid of a sprayer. Anhydrous dasatinib is dusted onto the thus-treated pellets, distributing the material manually as necessary.

In another illustrative embodiment both sugar nonpareils (mesh size 35-45) and MCC Spheres (Vivapure MCC spheres 200) were used. Spheres were selected that were of comparable starting particle size for the purposes of the coating.

In an illustrative embodiment the low dose coated nonpareils for use in pediatric patients are placed within capsules comprising a total effective therapeutic dose that a heath care professional can open and sprinkle the low dose coated nonpareils on food or suspend them within a solution or suspension allowing for solid or liquid suspension oral dosing in pediatric populations on a mg/kg basis.

In a further illustrative embodiment, the encapsulated low dose coated nonpareils for use in pediatric patients can be carded within a dose pack having multiple capsules having a single total dosage or multiple capsules having differing total dosages, so that the health care professional can dose the pediatric patient allowing for solid or liquid suspension oral dosing in pediatric populations on a mg/kg basis.

### BRIEF DESCRIPTION OF THE DRAWINGS

FIG. 1 is a graphic depiction of plasma dasatinib concentrations vs time comparing the drug delivery composition according to the invention with a reference formulation of dasatinib (20 mg IG-100 and Sprycel®).

### DETAILS OF THE INVENTION

Dasatinib was initially developed by Bristol-Myers Squib for the treatment of chronic phase, accelerated phase, or blastic phase Chronic Myeloid Leukemia (CML), resistant or intolerant to imatinib, and for Ph chromosome-positive acute lymphoid leukemia resistant or intolerant to prior therapy in adults (approved June 2006). More recently, FDA approved dasatinib for the treatment of pediatric patients aged one year or older with newly diagnosed Ph chromosome-positive acute lymphoblastic leukemia (approved November 2017).

A nonclinical study in mice was conducted to determine the PK and PD properties of a low-dose treatment of dasatinib in NS and NSML mice for the treatment of Hypertrophic cardiomyopathy (HCM). The goal was to establish in the same mouse model concurrent PK data and to correlate that with the endpoints of HCM using qPCR and immunoblotting. PK properties obtained from these mice were determined by Kana Mizuno and Alexander A. Vinks at Cincinnati Children's Hospital Medical Center. Dasatinib PK profiles and exposures (expressed as area under the concentration time curve; AUC) at doses ranging from 0.05 mg/kg to 0.5 mg/kg were estimated in NS and NSML mice. Based on the exposure-response relationship, potential target exposures to protect cardiac function were identified. Considering efficacy and safety, a suggested potential target AUC for cardiac rescue was identified at 12-24 ng·h/mL.

According to the invention, a pediatric dasatinib formulation (IG-100) is disclosed capable of being dosed at ultra-low dosages on a mg/kg basis. In one aspect of the invention a pediatric formulation is manufactured using a Wurster coating process on sugar nonpareils which are then coated with a water and HPMC seal coat. The final coated spheres demonstrate excellent uniformity and allow for solid or liquid suspension oral dosing in pediatric populations on a mg/kg basis.

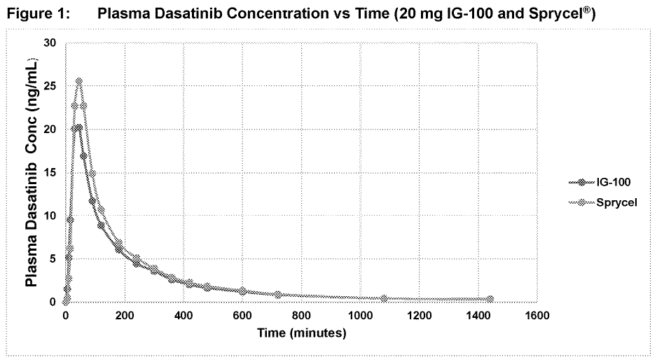

The pediatric formulation according to the invention was tested in a single-dose, open-label, randomized, two-period, two-treatment, crossover relative bioavailability study in 28 healthy adult subjects. Subjects received a single dose of IG-100 (20 mg suspension) in one period and a single dose of Sprycel® (20 mg tablet) in another period under fasted conditions. Human plasma samples were analyzed for dasatinib with a validated assay and plasma concentration data has been provided. As show in FIG. 1, the formulation according to the present invention indicates expected exposure profiles demonstrating bio-equivalence with that of the reference listed drug.

The development process for an ultra-low dosage form contemplates producing a suspension system that would allow approximately 1% of API (Dasatinib) onto the nonpareils. The suspension system is a base of water with hydroxypropyl cellulose as the binder along with some other excipients to increase the spraying properties. The API is charged into the suspension and continues to mix during the spraying process. The nonpareils are loaded into the fluid bed with a Wurster column (bottom spray); the beads are fluidized and warmed to approximately 60 °C. The spraying began with a goal of approximate weight gain that allows for about 1% active ingredient to be sprayed onto the nonpareils. The coating is sprayed at approximately 20 g/min. Once the weight gain is achieved the nonpareils are allowed to dry for approximately 15 mins. The beads are then screened through a 20-mesh screen to ensure no large agglomerates occurred.

The active coated beads were then loaded back into the fluid bed with the Wurster coater insert and a seal coat is applied. The seal coat is a combination of water and hydroxypropyl methylcellulose (HPMC) along with small number of excipients to allow for ease of coating. The seal coating process follows the same process with the exception to have 3-5% weight gain of solids on the active coated beads. The sugar versus MCC spheres do not have any impact. The final coated spheres have a uniformity of 95%-104% of the desired assay with an RSD of 3.7. According to one illustrative embodiment, the seal coated active beads contain approximately 7.6 mg dasatinib per 1 g of coated active beads.

The coated particle according to the invention comprises a suitable carrier, which is surrounded by a coating layer. Suitable carriers according to the invention, are preferably based on inert excipients, which are customarily used in formulation technology as carriers.

Excipients, which may preferably be used included but are not limited to the following: mannitol, saccharose, lactose (e.g. lactose monohydrate), glucose, erythritol, xylitol, cellulose, microcrystalline cellulose, starch, croscarmellose sodium, crospovidone and mixtures thereof. It is contemplated within the scope of the invention that other excipients known in the art may be used. It is further contemplated within the scope of the invention that carriers according to the invention may be based on powders, granules, small beads, particles, pellets, starter pellets, nonpareils of suitable size composed of the above excipients or mixtures thereof. If a defined shape (e.g. round shape) of the coated particles is desired, it is advantageous to use carriers of a defined shape and size such as starter pellets made of microcrystalline cellulose or saccharose (nonpareilles).

Although the above illustrative embodiment utilizes the anhydrous dasatinib, one skilled in the art will appreciate that the method and compositions used in the manufacturing of the pediatric formulation can be used for other TKIs. It is contemplated within the scope of the invention that other TKIs include but are not limited to the following TKIs: afatinib, axitinib, bosutinib, cabozantinib, cediranib, ceritinib, crizotinib, dabrafenib, dasatinib, erlotinib, everolimus, gefitinib, ibrutinib, imatinib, lapatinib, lenvatinib, lestaurtinib, nilotinib, nintedanib, palbociclib, pazopanib, ponatinib. It is further contemplated within the scope of the invention that these TKIs may be used alone or in combination with each other or in combination with other APIs that would be beneficial to a desired therapeutic effect. Likewise, it should be appreciated by those skilled in the art that the TKIs may be utilized in a variety of salt forms, analogues and prodrugs thereof.

It will be appreciated that the actual dosages of the agents used in the compositions of this invention will vary according to the particular complex being used, the particular composition formulated, the mode of administration and the particular site, host and disease and/or condition being treated. Actual dosage levels of the active ingredient(s) in the pharmaceutical compositions of the present invention can be varied so as to obtain an amount of the active ingredient which is effective to achieve the desired therapeutic response for a particular subject, composition, and mode of administration, thereby providing a therapeutic amount without being toxic to the human subject.

The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000.

Optimal dosages for a given set of conditions can be ascertained by those skilled in the art using conventional dosage-determination tests view of the experimental data for an agent. For oral administration of the formulation according to the invention an exemplary daily dose generally employed is from about 0.001 to about 3000 mg/kg of body weight, with courses of treatment repeated at appropriate intervals. In some embodiments, the daily dose is from about 1 to 3000 mg/kg of body weight. Methods and compositions according to the present invention are suitable for use in treating diseases and conditions of both humans and non-humans, including treatment of socially and economically important animals such as dogs, cats, cows, horses, sheep, pigs, goats, and other species. Unless specified, methods and compositions according to the present invention are not limited to treatment of humans.

Typical daily doses in a patient may be anywhere between about 0.04 mg/kg to about 0.22 mg/kg, given once daily or in divided doses. In one embodiment, the dose is between about 0.14 mg to about 1.1 mg. In another embodiment, the dose is between about 0.2 mg to about 1.7 mg. In other embodiments, the dose is between about 0.24 mg to about 2.4 mg.

In particular, for dasatinib or derivatives or analogs thereof, suitable doses typically are from about 0.04 mg mg/kg to about 0.22 mg/kg as shown in Table 1 set forth below

TABLE I
PK Model-based Optimal IG-100 Dosing in Pediatric Patients
with NS or NSML to Achieve Different Exposure Targets

| | Cohort 1 | | Cohort 2 | | Cohort 3 | |
|---|---|---|---|---|---|---|
| | Target | | Target | | Target | |
| | AUC 12-24 | | AUC 12-61 | | AUC 12-121 | |
| | ng · h/mL | | ng · h/mL | | ng · h/mL | |
| | IG-100 | IG-100 | IG-100 | IG-100 | IG-100 | IG-100 |
| Age | Dose | Dose¹ | Dose | Dose¹ | Dose | Dose¹ |
| (months) | (mg/kg) | (mg) | (mg/kg) | (mg) | (mg/kg) | (mg) |
| 0-2 | 0.04 | .14 | 0.06 | 0.2 | 0.08 | 0.24 |
| 2-6 | 0.06 | 0.4 | 0.10 | 0.7 | 0.13 | 0.9 |
| 6-12 | 0.08 | 0.7 | 0.13 | 1.1 | 0.18 | 1.6 |
| 12-24 | 0.10 | 1.1 | 0.16 | 1.7 | 0.22 | 2.4 |

¹IG-100 dose is based on average infant weight (50^{th} percentile) for each pediatric age range derived from the World Health Organization (WHO) growth charts for the first two year of life (0 to 2 years)

Pharmaceutical preparations for oral use can be obtained by combining the pharmacologically active agent with solid excipients as set forth within this disclosure. Suitable excipients are, in particular, fillers including but are not limited to the following: sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP)

If desired, disintegrating modulators may be added, such as the cross-linked polyvinyl pyrrolidone, agar, or alginic acid or a salt thereof such as sodium alginate. Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-a-naphthylamine, or lecithin can be used.

Also, other ingredients that are conventional in the area of pharmaceutical compositions and formulations, such as lubricants, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients well known in the art are contemplated within the scope of the invention. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA and other regulatory agencies.

Pharmaceutical compositions according to the present invention can be prepared in accordance with methods well known and routinely practiced in the art. See, e.g., Remington: The Science and Practice of Pharmacy, Mack Publishing Co., 20th ed., 2000. Pharmaceutical compositions are preferably manufactured under GMP conditions. Pharmaceutical compositions according to the present invention are usually administered to the subjects on multiple occasions. Intervals between single dosages can be daily, weekly, monthly or yearly. Intervals can also be irregular as indicated by therapeutic response or other parameters well known in the art.

Alternatively, the pharmaceutical composition can be administered as a sustained release formulation, in which case less frequent administration is required. Dosage and frequency vary depending on the half-life in the subject of the pharmacologically active agent included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives.

It is contemplated within the scope of this invention that sustained-release formulations or controlled-release formulations that are well-known in the art may be utilized to deliver the active ingredients as set forth above. The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising a pharmacologically active agent according to the present invention by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the pharmacologically active agent, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the pharmacologically active agent. Accordingly, one of ordinary skill in the art could modify the formulations into a formulation having the desirable properties described above for a particular application.

### EXAMPLES

The following examples are provided to illustrate certain features of the present invention. These examples should not be construed to limit the present invention to the particular features stated in these examples.

### Example I Development Process

Major Equipment List

| Equipment | Sterling Equipment ID # |
|---|---|
| Scale | SPS-001 |
| Scale | SPS-002 |
| Caframo Stir Machine | SPS-0031 |

### Material Formula

$$\frac{250. g}{( Total sugar spheres )} \times 1.8571 = \frac{{464.275}^{4}}{\begin{matrix}( Theoretical end weight of \\ coated sugar spheres )\end{matrix}}$$

Theoretical End Weight

| | Per 130 | | % | | Quantity |
|---|---|---|---|---|---|
| Raw Material | mg (mg) | % | Excess | % | per Batch |
| Sugar Sphere | 46.5 | 35.769 | — | 35.769 | |
| 180-250 μm | | | | | |
| Sugar Sphere | 23.5 | 18.077 | — | 18.077 | |
| 425-500 μm | | | | | |
| Hypromellose | 15.0 | 11.538 | 20.0 | 13.846 | ¹ |
| Ammonia Solution | — | — | — | — | — |
| Strong | | | | | |
| Talcum | 15.0 | 11.538 | 20.0 | 13.846 | ⁶ |
| | 30.0 | 23.077 | 20.0 | 27.692 | ⁷ |
| Water⁵ | — | — | — | — | — |
| Total | 130.0 | 100.0% | — | — | 464.275 ⁴ |

⁵Evaporates during coating process

### Coating

$$\frac{1}{{( Quantity per Batch )}^{1}} / \frac{2}{{( \% of coating suspension )}^{2}} = \frac{3}{\begin{matrix}( Theoretical end \\ {weight of coating )}^{3}\end{matrix}}$$

| | | Quantity |
|---|---|---|
| Raw Material | % | per Coating |
| Hypromellose | 3.7413² | ¹ |
| Ammonia Solution Strong | 0.0611 | |
| Talcum | 3.7413 | ⁶ |
| Dasantinib | 1.00 | ⁷ |
| Water | 92.456 | |
| Total | 100.0 | ³ |

Theoretical End Weight

| | | | | | | | |
|---|---|---|---|---|---|---|---|
| Raw | Material | Material | Tare | Gross | Net | Performed | Checked |
| Material | Lot # | Supplier | Weight | Weight | Weight | By/Date | By/Date |

| | | | | | | |
|---|---|---|---|---|---|---|
| Sugar Sphere | G2781 | Paulaur | 25.0 | 191.1 | 166.1 | g |
| 180-250 μm | | | | | | |
| Sugar Sphere | Z6945 | Paulaur | 25.0 | 108.9 | 83.9 | g |
| 425-500 μm | | | | | | |
| Hypromellose | IHD0862 | Dupont | 30.0 | 94.3 | 64.3 | g |
| Ammonia | 1189 | Spectrum | — | 1,050 μL | 1,050 | μL |
| Solution Strong | | | | | | |
| Talcum | 2DA0307 | Barrett | 30.0 | 94.3 | 64.3 | g |
| Dasantinib | 7SM20017423616 | Teva | 10.0 | 11.9 | 1.9 | g |
| Water | NA | SPS | — | 1,460.0 | 1,460.0 | g |

### Manufacturing Instructions

1. Mix Water at about 450 RPM.
2. Add Hypromellose and mix for about 45 minutes, or until solution is clear.
3. Add Ammonia Solution Strong and mix for about 10 minutes.
4. Add Talcum and mix for about 10 minutes.
5. Increase speed to about 850 RPM and slowly add Dasatinib. Mix for about 15 minutes.
6. Reduce the speed to about 300 RPM and mix for at least 2 hours.
7. Wurster coat Dasatinib with approximately 75% of coating suspension.

Major Equipment List

| Equipment | Sterling Equipment ID # |
|---|---|
| Fluid Bed, Wurster | SPS-0009 |
| Nozzle—0.8 mm | |
| Caframo Stir Machine | SPS-0031 |
| Fluid Aid Pump | SPS-0046 |

Major Equipment List

| Equipment | Sterling Equipment ID # |
|---|---|
| Scale | SPS-001 |
| Scale | SPS-002 |
| Caframo Stir Machine | SPS-0031 |

### Material Formula

$$\frac{250. {g}^{5}}{{( Total drug coated pellets )}^{2}} \times 1.0769 = \begin{matrix}269.22 {5}^{4} \\ \begin{matrix}( Theoretical end weight \\ of drug coated pellets\end{matrix}\end{matrix}$$

Theoretical End Weight

| | Per 1 mg | | % | | Quantity |
|---|---|---|---|---|---|
| Raw Material | (mg) | % | Excess | % | per Batch |
| Drug Coated | 130.0 | 92.857 | — | 92.857 | ⁵ |
| Pellets | | | | | |
| Hypromellose | 4.0 | 2.8571 | 30.0 | 5.2002 | ¹ |
| Talcum | 6.0 | 4.2857 | 30.0 | 7.8003 | ⁷ |
| Water⁶ | — | — | — | — | — |
| Total | 140.0 | 100.0% | — | — | ⁴ |

⁶Evaporates during coating process

### Coating

$$\frac{1}{{( Quantity per Batch )}^{1}} / \frac{2}{{( \% of coating suspension )}^{2}} = \frac{3}{\begin{matrix}( Theoretical end \\ {weight of coating )}^{3}\end{matrix}}$$

| | | Quantity |
|---|---|---|
| Raw Material | % | per Coating |
| Hypromellose | 6.0095² | ¹ |
| Talcum | 9.0143 | ⁷ |
| Water | 84.976 | |
| Total | 100.0 | ³ |

Theoretical End Weight

| | | | | | | | |
|---|---|---|---|---|---|---|---|
| Raw | Material | Material | Tare | Gross | Net | Performed | Checked |
| Material | Lot # | Supplier | Weight | Weight | Weight | By/Date | By/Date |

| | | | | | |
|---|---|---|---|---|---|
| Drug Coated | RTF 138 | SPS | — | 250.0 g | 250.0 g |
| Pellets | | | | | |
| Hypromellose | IHD0862 | Dupont | 30.0 | 44.0 g | 14.0 g |
| Talcum | 2DA0307 | Barrett | 30.0 | 51.0 g | 21.0 g |
| Water | NA | SPS | — | 198.0 g | 198.0 g |

### Manufacturing Instructions

1. Mix Water at about 450 RPM.
2. Add Hypromellose and mix for about 45 minutes, or until solution is clear.
3. Add Talcum and mix for about 10 minutes.
4. Reduce the speed to 300 RPM and mix for at least 1 hours.
5. Wurster coat with approximately 70% of coating suspension.

Major Equipment List

| Equipment | Sterling Equipment ID # |
|---|---|
| Fluid Bed, Wurster | SPS-0009 |
| Nozzle—0.8 mm | |
| Caframo Stir Machine | SPS-0031 |
| Fluid Aid Pump | SPS-0046 |

| | Inlet | Inlet | Outlet | Spray Air | Pump |
|---|---|---|---|---|---|
| | Flow | Temp. | Temp. | Pressure | Speed |
| Time | (SCFM) | (°C) | (°C) | (PSI) | (%) |
| 09:40 | — | 55 | 31.7 | — | — |
| 09:52 | 14 | 55 | 32.6 | 15 | 10 |
| 10:05 | 15 | 55 | 30.9 | 20 | 08 |
| 10:15 | 15 | 55 | 31.9 | 21 | 11 |
| 10:30 | 15 | 56 | 31.6 | 21 | 10 |
| 10:40 | 15 | 56 | 31.8 | 21 | 11 |
| 10:50 | 17 | 56 | 36 | 15 | — |

### Example II Bioavailability Study Comparing Dasatinib 20 mg Tablets and IG-100 20 mg Suspension Under Fasted Conditions.”

### Study Information

Samples were collected in support of “A Pilot Single-Dose, Two-Period, Two-Treatment, Two-Way Crossover Relative Bioavailability Study Comparing Dasatinib 20 mg Tablets and IG-100 20 mg Suspension under Fasted Conditions.” The samples were analyzed according to Worldwide-SOP-BSC-006, Revision 4, and processed according to Worldwide-SOP-BSC-011, Revision 6. Study data were collected using Analyst® (Version 1.6.1, Applied Biosystems/Sciex) and Watson Laboratory Information Management System™ (LIMS; Version 7.5, Thermo Fisher Scientific) software.

### Method Summary

Human plasma samples were analyzed for dasatinib according to Worldwide procedure ATM-2572, Original, effective 23 Oct. 2020. The assay validation was finalized and reported under Worldwide DCN 1004600. The method used in this study was validated for a range of 0.200 to 200 ng/ml based on the analysis of 0.0500 mL of plasma by LC-MS-MS. Quantitation was performed using a weighted 1/x² linear least squares regression analysis generated from calibration standards.

### Preparation of Solutions

Stock solutions were prepared and stored according to the analytical test method instructions as presented in Table II.

TABLE II

| Analyte/ | Target | | Preparation | Storage | Storage | Stock |
|---|---|---|---|---|---|---|
| Internal | Concentration | Solvent | Date | Container | Temperature | Stability |
| Dasatinib | 200 μg/mL | Dimethyl | 1 Sep. 2020; | Polypropylene | −20 °C | 42 Days |
| | | Sulfoxide/Methanol/ | 28 Sep. 2020; | | 22 °C | 25 Hours |
| | | Acetic | 13 Oct. 2020; | | | |
| | | Acid; 50:50:0.2 | 17 Nov. 2020 | | | |
| Dasatinib- | 100 μg/mL | Dimethyl | 12 Oct. 2020 | Polypropylene | −20 °C | 42 Days |
| D8 | | Sulfoxide/Methanol/ | | | 22 °C | 25 Hours |
| | | Acetic | | | | |
| | | Acid; 50:50:0.2 | | | | |

Solutions, calibration standards, and quality control samples were prepared using the following reference materials. Certificates of analysis are provided in the Reference Material Certificates of Analysis section of this report and may include only the textual portions.

| | Reference | Internal |
|---|---|---|
| IGIA Pharmaceuticals, | Standard(s) | Standard(s) |
| Compound | Dasatinib | Dasatinib-d8 |
| Source | Toronto Research | Toronto Research |
| | Chemicals | Chemicals |
| Lot/Batch | 2-OBI-71-1 | 3-JES-141-4 |
| Purity | 98.0% | N/A |
| Storage Condition | −20 °C | −20 °C |
| Expiration Date | 6 May 2023 | 25 Oct. 2023 |

Solutions, calibration standards, quality controls, and matrix control blanks were prepared using the following matrices as shown in Table III.

TABLE III
Human K2-EDTA Plasma

| Lot(s) | Receipt Date | Vendor |
|---|---|---|
| HMN345073 | 26 Feb. 2020 | BioIVT |
| HMN345075 | 26 Feb. 2020 | BioIVT |
| HMN368210 | 2 Apr. 2020 | BioIVT |
| HMN368216 | 2 Apr. 2020 | BioIVT |
| HMN405439 | 3 Jun. 2020 | BioIVT |
| HMN442300 | 6 Aug. 2020 | BioIVT |
| HMN442301 | 6 Aug. 2020 | BioIVT |
| HMN460790 | 11 Sep. 2020 | BioIVT |
| HMN460793 | 11 Sep. 2020 | BioIVT |
| HMN460794 | 11 Sep. 2020 | BioIVT |
| HMN460795 | 11 Sep. 2020 | BioIVT |
| HMN478211 | 8 Oct. 2020 | BioIVT |
| HMN478213 | 8 Oct. 2020 | BioIVT |

### Standard Curves

The peak area ratios (y) of dasatinib to the internal standard and the concentrations of the calibration standards (x) were fitted by a weighted linear least squares regression analysis to the equation y=a+bx, where “a” is the y-intercept and “b” is the slope of the calibration curve. Calibration standards used for each of the sample sets were entered as unknowns into the derived equation of the least squares regression line to obtain “back-calculated” values.

Calibration standards were prepared at 0.200, 0.400, 2.00, 10.0, 20.0, 100, 180 and 200 ng/ml for dasatinib in human K2-EDTA plasma on the day of each extraction, see Individual Run Report, Table IV) and aliquoted out at 0.0500 mL.

TABLE IV
Individual Run Report Selection for Study - 4009179

| Run | Analyte | Regression | Run | Assay | | Biological | Extraction | Assay | LLOQ | ULOQ | Regression |
|---|---|---|---|---|---|---|---|---|---|---|---|
| ID¹ | Name | Status | Type | Name | Species | Matrix | Date | Date | (ng/mL) | (ng/m | Type |
| 1 | Dasatinib | Accept | UNKNOWN | 2572r | Hum | K2-EDTA | 27-Oct- | 28-Oct- | 0.2 | 200 | Linear |
| 2 | Dasatinib | Accept | UNKNOWN | 2572r | Hum | K2-EDTA | 28-Oct- | 29-Oct- | 0.2 | 200 | Linear |
| 3 | Dasatinib | Accept | UNKNOWN | 2572r | Hum | K2-EDTA | 02-Nov- | 02-Nov- | 0.2 | 200 | Linear |
| 4 | Dasatinib | Accept | UNKNOWN | 2572r | Hum | K2-EDTA | 27-Oct- | 27-Oct- | 0.2 | 200 | Linear |
| 5 | Dasatinib | Accept | UNKNOWN | 2572r | Hum | K2-EDTA | 03-Nov- | 03-Nov- | 0.2 | 200 | Linear |
| 6 | Dasatinib | Accept | UNKNOWN | 2572r | Hum | K2-EDTA | 03-Nov- | 04-Nov- | 0.2 | 200 | Linear |
| 7 | Dasatinib | Accept | UNKNOWN | 2572r | Hum | K2-EDTA | 06-Nov- | 06-Nov- | 0.2 | 200 | Linear |
| 8 | Dasatinib | Accept | UNKNOWN | 2572r | Hum | K2-EDTA | 30-Nov- | 01-Dec- | 0.2 | 200 | Linear |
| 9 | Dasatinib | Accept | UNKNOWN | 2572r | Hum | K2-EDTA | 30-Nov- | 30-Nov- | 0.2 | 200 | Linear |
| 10 | Dasatinib | Accepted | UNKNOWNS | 2572r Org | Human | K2-EDTA | 03 Dec. | 04 Dec. | 0.2 | 200 | Linear |
| | | | | | | Plasma | 2020 | 2020 | | | |

### Quality Control Samples

Qualifying quality control (QC) samples were prepared as shown in the table below and processed along with each study run. Sample runs were considered valid when at least two-thirds of the qualifying QC samples were within 15% of their theoretical values and at least 50% of the QCs at each level met this criterion. Additionally, if sample runs exceeded the capacity of one 96 well block, each block was evaluated individually and considered valid when at least two-thirds of the qualifying QC samples were within 15% of their theoretical values (after QCs were deactivated for cause as applicable) and at least 50% of the QC samples at each level met this criterion. If an individual block within a run did not meet acceptance criteria, all samples within that block were repeated as analytical repeats. The calculated QC values were recorded for monitoring of the precision and accuracy of the assay.

### Sample Analysis

Between 21 Oct. 2020 and 19 Nov. 2020, samples were received at Worldwide (including duplicate samples processed at the clinic and shipped to the bioanalytical facility). Samples were logged in and stored at approximately −20 °C in boxes clearly labeled with Worldwide shipment IDs in Table V.

TABLE V

| | Concentration | | | |
|---|---|---|---|---|
| | of Dasatinib | Date | Storage | Storage |
| QC Level | (ng/mL) | Prepared | Container | Temperature |
| High | 160 | 3 Sep. 2020 | Polypropylene | −20 °C |
| Mid-High | 80.0 | 29 Sep. 2020 | | |
| Medium | 10.0 | 23 Oct. 2020 | | |
| Low | 0.600 | 3 Sep. 2020 | | |

### Sample Collection Dates and Storage Term

Sample storage exceeded the established long-term stability of 15 days at −20 °C for dasatinib. Additional long-term stability for at least 48 days at −20 °C is pending for dasatinib in human K2-EDTA.

| Date of First | Date of Last Sample | Duration of |
|---|---|---|
| Sample Collection | Analysis (including ISR) | Sample Storage |
| 16 Oct. 2020 | 3 Dec. 2020 | 48 days |

### Incurred Sample Reproducibility

Incurred sample reproducibility (ISR) for dasatinib was evaluated for clinical samples using ATM-2572, Original. Select incurred samples near the Cmax and within the elimination phase for IGIA Pharmaceuticals, Inc. Protocol 4009179 Bioanalytical Study Report at least 10% of the samples across subjects were reanalyzed in singlicate. ISR is demonstrated if at least two-thirds overall of the concentrations obtained for the original and repeat analysis deviate no more than ±20.0% of their mean concentration. The column denoting this % variability calculation is labeled as “% Bias.” Due to Watson limitations, the naming convention cannot be updated to “% Variability.” Samples outside individual ISR acceptance criteria, if applicable, are reported with a “>20.0” notation within the Flag column. Sample results were excluded from the ISR evaluation if the reassay result was marked as not reportable (NR). In such instances the percent variability could not be determined, and an asterisk is reported in the % Bias column.

The ISR data are provided in the Incurred Sample Repeats Report, Table VI.

TABLE VI

| Number of | Evaluable | Number of | Number of | Percent |
|---|---|---|---|---|
| Samples | Results | Samples | Samples | Acceptable |
| 103 | 101 | 100 | 1 | 99.0% |

### Run Restarts and Reinjections

No runs in this study required a restart.

One of the 10 total runs within this study required at least one reinjection.

The following run was reinjected for the indicated reasons. The appropriate corrective actions were taken to resolve the issue prior to each reinjection.

TABLE VII

| Reinjected | | |
|---|---|---|
| Run Number | Reason | Resolution |
| 10 | Carryover: Carryover was | The instrument was changed. |
| | observed. | |
| 10 | Identifiable injection error: | The column was changed. |
| | the peak was out | |

### Reimported Runs

No runs were reimported within the study.

### Rejected Runs

All data within the study reported herein were acceptable.

### Deviations

No method deviations that impacted the integrity of the data occurred during the study. All protocol and SOP deviations that occurred are on file with the study and had no impact on the integrity of the data. In Run 1, the Scientist inadvertently failed to start the run with a QC low after the carryover blank and failed to start the second block with a QC low. All standards, curve, and QCs were within range and the run met acceptance criteria. Data was not impacted.

In Runs 5 and 6, the Validation revision of ATM-2572 was inadvertently used. The Validation revision was compared to the Original revision to confirm no changes had been made that would affect sample analysis. Data was not impacted.

The QC Mid-High batch was qualified on Day 24 in ATM257204, when only 15 days of LTS at −20 °C had been established for dasatinib. The QC validation run met acceptance criteria and sufficient LTS (104 days) was established to cover all study samples and prepared QC samples. According to the study protocol, only subjects completing at least one period were to be analyzed. Samples from Subject 115 were inadvertently analyzed despite not completing Period 1, Treatment A, Day 1, 18 hour and Period 1, Treatment A, Day 2, 24 hour, resulting in a protocol deviation. All Subject 115 samples that were analyzed were marked as NR and are not included on the tables.

### CONCLUSION

The pediatric formulation according to the invention in an open-label, randomized, two-period, two-treatment, crossover relative bioavailability study in 28 healthy adult subjects indicates expected exposure profiles demonstrating bio-equivalence with that of the reference listed drug as shown in FIG. 1.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional

features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference. While we have described and given examples of preferred embodiments of our inventions, it will be apparent to those of ordinary skill in the art that changes and modifications may be made without departing from our inventions in their broader aspects. We therefore intend the appended claims to cover all such changes and modifications as fall within the true spirit and scope of our invention.

What is claimed:

1. A pediatric dosage form for dosing ultra-low doses of an active pharmaceutical ingredient comprising a plurality of pellets where each pellet consists essentially of a coating of tyrosine kinase inhibitor over a nonpareil seed, the tyrosine kinase inhibitor coated nonpareils are coated thereon with a coating consisting essentially of hydroxypropyl methylcellulose, the sum of such pellets in forming a single dosage unit of an ultra-low dose of tyrosine kinase inhibitor.

2. The ultra-low dosage form of claim 1 wherein said tyrosine kinase inhibitor is selected from the group consisting of afatinib, axitinib, bosutinib, cabozantinib, cediranib, ceritinib, crizotinib, dabrafenib, dasatinib, erlotinib, everolimus, gefitinib, ibrutinib, imatinib, lapatinib, lenvatinib, lestaurtinib, nilotinib, nintedanib, palbociclib, pazopanib and ponatinib.

3. The ultra-low dosage form of claim 1 wherein said tyrosine kinase inhibitor is a combination of one or more tyrosine kinase inhibitors selected from the group consisting of afatinib, axitinib, bosutinib, cabozantinib, cediranib, ceritinib, crizotinib, dabrafenib, dasatinib, erlotinib, everolimus, gefitinib, ibrutinib, imatinib, lapatinib, lenvatinib, lestaurtinib, nilotinib, nintedanib, palbociclib, pazopanib and ponatinib.

4. The ultra-low dosage form of claim 1 wherein said tyrosine kinase inhibitor is dastinib.

5. The ultra-low dosage form of claim 3 wherein the dastinib is in an anhydrous form.

6. The ultra-low dosage form of claim 1, wherein nonpareil seeds are coated with more than an equal amount by weight of said tyrosine kinase inhibitor.

7. The ultra-low dosage form of claim 1, wherein approximately 1% of said tyrosine kinase inhibitor is coated onto the nonpareils.

8. The ultra-low dosage form of claim 1, wherein said pellets contain approximately 7.6 mg dasatinib per 1 g of coated spheres.

9. The ultra-low dosage form of claim 1, wherein said sum of such pellets are encapsulated in capsules having a desired therapeutic dose.

10. The ultra-low dosage form of claim 9, wherein said capsules are carded onto a dose-pack containing a singular therapeutic dose.

11. The ultra-low dosage form of claim 9, wherein said capsules are carded onto a dose-pack containing a multiple therapeutic doses.

12. A method of formulating an ultra-low dosage form for pediatric treatment comprising the following steps,
loading nonpareils into a fluid bed with a Wurster column, wherein the nonpareils are fluidized and warmed to approximately 60 °C;
spraying said nonpareils with a coating of an active pharmaceutical ingredient within a suspension allowing for a desired weight gain of said nonpareils;
drying said nonpareils coated with said active pharmaceutical ingredient forming active coated bead;
screening said active coated beads;
loading said active coated bead into a fluid bed with the Wurster coater insert; and
applying a seal coat to said active coated beads to achieve a desired weight gain.

13. The method of formulating an ultra-low dosage form for pediatric treatment of claim 12, wherein said nonpareils are sugar nonpareils having a mesh size of 35-45.

14. The method of formulating an ultra-low dosage form for pediatric treatment of claim 12, wherein said active pharmaceutical ingredient is a tyrosine kinase inhibitor.

15. The method of formulating an ultra-low dosage form for pediatric treatment of claim 14, wherein said tyrosine kinase inhibitor is selected from the group consisting of afatinib, axitinib, bosutinib, cabozantinib, cediranib, ceritinib, crizotinib, dabrafenib, dasatinib, erlotinib, everolimus, gefitinib, ibrutinib, imatinib, lapatinib, lenvatinib, lestaurtinib, nilotinib, nintedanib, palbociclib, pazopanib and ponatinib.

16. The method of formulating an ultra-low dosage form for pediatric treatment of claim 14, wherein said tyrosine kinase inhibitor is dasatinib.

17. The method of formulating an ultra-low dosage form for pediatric treatment of claim 12, wherein said nonpareils are microcrystalline cellulose.

18. The method of formulating an ultra-low dosage form for pediatric treatment of claim 12, wherein said active pharmaceutical ingredient within a suspension is approximately 1 percent dasatinib.

19. The method of formulating an ultra-low dosage form for pediatric treatment of claim 12, wherein said seal coat is water, hydroxypropyl methylcellulose and excipients.

20. The method of formulating an ultra-low dosage form for pediatric treatment of claim 12, wherein said seal coated active coated beads allows for human subject dosing on a mg/kg basis.
